(19) United States
(12) Patent Application Publication
(10) Pub. No.: US 2019/0060645 A1
(43) Pub. Date: Feb. 28, 2019

(54) WIRELESS BONE STIMULATOR SYSTEM AND METHOD

(71) Applicant: GLOBUS MEDICAL, INC., AUDUBON, PA (US)

(72) Inventor: Brian Kearns, Moorestown, NJ (US)

(21) Appl. No.: 15/691,095

(22) Filed: Aug. 30, 2017

(51) Int. Cl. A61N 1/32; A61N 1/372; A61N 1/05

(52) U.S. Cl. CPC A61N 1/326; A61N 1/08; A61N 1/05; A61N 1/37211

(57) ABSTRACT

A bone stimulator system contains an implantable electrical implant, such as an electrode having a receiver, that communicates with an external pulse generator generating electromagnetic stimulation signals to aid in a bone's healing process. The electrical implant is implanted at or near a target area, such as a broken bone or a fusion site. The bone stimulator system may also contain an external programmer used by the patient in order to select certain therapy parameters for pulse generator to deliver to the electrical implant.

### FIELD

This disclosure relates to implantable stimulators used to provide electromagnetic energy to targeted areas within the body, and more particularly, applying electromagnetic energy to an injured bone or fusion site to stimulate the bone's natural healing process.

### BACKGROUND OF THE INVENTION

Stimulation systems for aiding bone growth typically involve sending electrical signals or pulses to the affected bone or area. These bone stimulation systems are sometimes used for patients that have issues in the bone healing process after a fracture, injury, or in other instances necessitating bone fusion. For example, patients that undergo spinal fusion surgery to fuse two adjacent vertebrae may have various complications that may prevent the vertebrae from fusing and a bone growth stimulator may be used in order to facilitate that healing and fusion process.

Presently, there are external wearable systems that transmit electromagnetic energy or pulses to the affected area. Disadvantages of this type of system include that the system is cumbersome and bulky and may be a relative significant distance from the affected area. Other types of implantable stimulation systems, not necessarily limited to bone stimulators, may include an implantable pulse generator with an internal battery, leads, and electrodes that are attached at or near the affected area. Disadvantages of these types of systems include the invasive nature of the implantable device, leads, and electrodes and the complicated surgery required in order implant the device. Other types of systems may include implantable leads and electrodes that are physically connected to an external programmer and power source. These types of systems may suffer from the disadvantage of leads that need to exit the body in order to connect with the external programmer and power source.

There exists, therefore, a need for a system and method that is non or minimally invasive for delivering bone stimulation therapy, such as wireless transmission of electromagnetic energy to an implantable receiver placed on or adjacent to the injured bone. There may also exist a need for delivering therapy to a receiver located on a medical device implant that is used to repair the bone injury. The receiver may be designed to be much less invasive than implantable pulse generator. Bone stimulation resulting from sending wireless low-level pulses of electromagnetic energy to the injury or fusion site may result in improved bone growth compared to currently used treatments and would be less invasive and/or cumbersome than existing systems and methods.

### SUMMARY OF THE INVENTION

A bone stimulation system includes an electrical implant having a receiver and a pulse generator system having a pulse generator and a pulse generator antenna. The pulse generator is in wireless communication with the receiver via the pulse generator antenna. The bone stimulation system also includes a programmer having a programmer antenna. The programmer is in wireless communication with the pulse generator system via the programmer antenna and the pulse generator antenna. The pulse generator system provides electromagnetic signals to the electrical implant via communication between the pulse generator antenna and the receiver.

A bone stimulation system includes an implantable electrode having a receiver and an external pulse generator system having a pulse generator, a processor, a pulse generator antenna, and a battery. The pulse generator is in wireless communication with the receiver via the pulse generator antenna. The bone stimulation system also includes an external programmer having a programmer antenna. The programmer is in wireless communication with the pulse generator system via the programmer antenna and the pulse generator antenna. The programmer comprises one or more selectable therapy programs and wherein the pulse generator system provides electromagnetic signals representative of a selected therapy program to the implantable electrode via communication between the pulse generator antenna and the receiver.

### BRIEF DESCRIPTION OF THE DRAWINGS

FIG. 1 illustrates an exemplary implantable spinal cord stimulator.

FIG. 2 illustrates an exemplary spinal cord stimulator with an external pulse generator.

FIG. 3 illustrates an exemplary bone stimulator system.

FIG. 4 illustrates an exemplary bone stimulator system consistent with the principles of the present disclosure.

FIG. 5 depicts an exemplary case for housing components of the bone stimulator system consistent with the present disclosure.

### DETAILED DESCRIPTION OF THE INVENTION

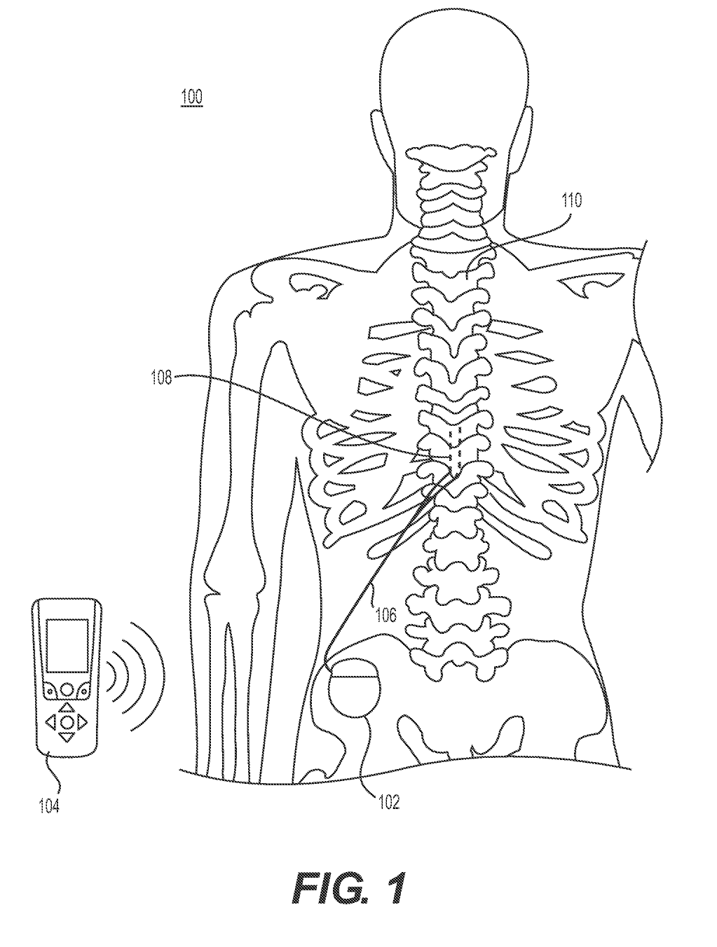

FIG. 1 depicts an exemplary spinal cord stimulator 100 known to those of ordinary skill in the art. Spinal cord stimulator 100 may comprise an implantable pulse generator 102 in communication with an external programmer 104. Implantable pulse generator 102 may electrically connect to leads 106, which in turn are electrically connected to electrodes 108. As known in the art, a user may select certain therapy parameters on external programmer 104 which are wirelessly communicated to implantable pulse generator 102. Implantable pulse generator 102 may then deliver electrical stimulation pulses to leads 106 that ultimately go to electrodes 108 that are housed near the therapy target area of spine 110. A feature of spinal cord stimulator 100 is that the components delivering the stimulation are completely implantable within a patient. The downside, however, is that this may involve complicated surgery that may not be suitable for all patients.

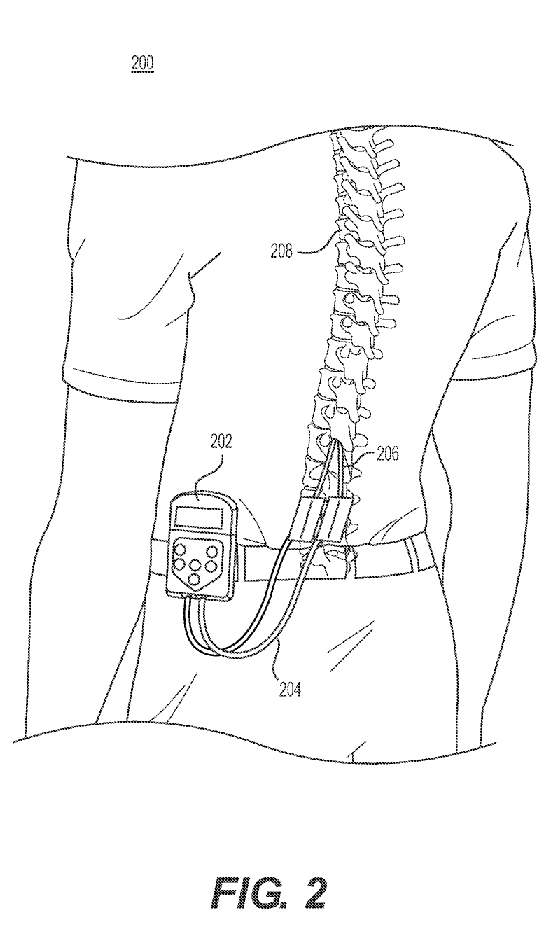

FIG. 2 depicts another type of spinal cord stimulator system 200. In system 200, a housing 202, which may contain both a pulse generator and a programmer, may not be implanted into a patient. Rather, these components may be disposed outside of the patient, with direct wired connections to leads 204, which are then connected to electrodes 206. The user may select therapy programs from housing 202. And these programs are used by the pulse generator to deliver electrical simulation pulses to a therapy target center of spine 208. System 200 may have certain characteristics that may be useful for some patients, including minimizing the components that are implanted into the patient. Here, only electrodes 206 and leads 204 reside inside the patient. This may lead to some disadvantages, however, such as the leads 204 physically exiting the body in order to connect with housing 202. This may lead to potential negative consequences affecting the patient's health and safety.

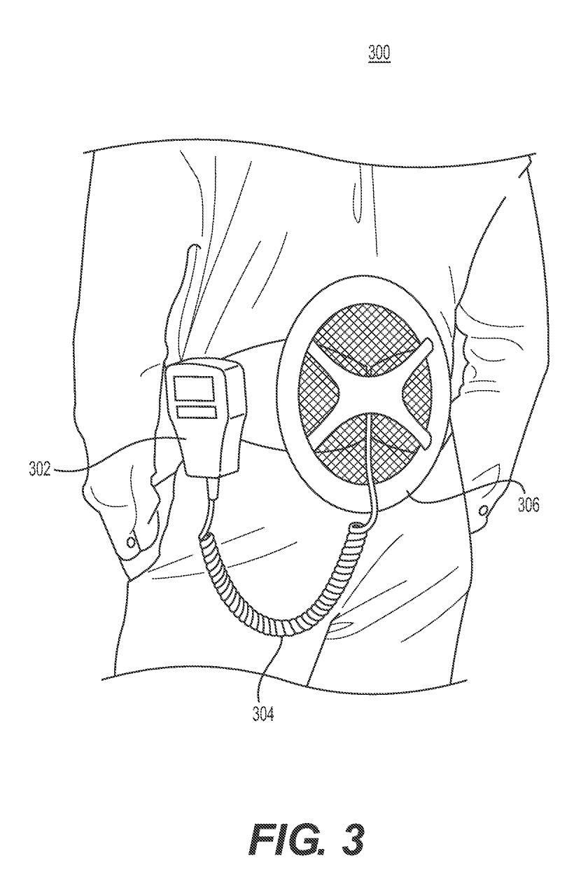

FIG. 3 depicts an exemplary bone stimulator that is currently known to those of ordinary skill in the art. Bone stimulator system 300 includes an external programmer 302, a cord 304, and external therapy device 306. In operation, a user chooses a therapy program using external programmer 302 which communicates with external device 306, via cord 304, to deliver electromagnetic signals or pulses to the affected area. In the example of FIG. 3, the user may have had spinal fusion surgery affecting adjacent vertebrae in the user's lumbar region. Bone stimulator 300 may deliver electromagnetic therapy to that site in order aid in the bone's healing process to promote fusion. While these types of bone stimulator exist, they suffer from numerous disadvantages. For example, users may find the system bulky and cumbersome and the source of the electromagnetic therapy (for example, external device 306), may not be close enough to the target bone(s) in order to maximize the benefits of the therapy.

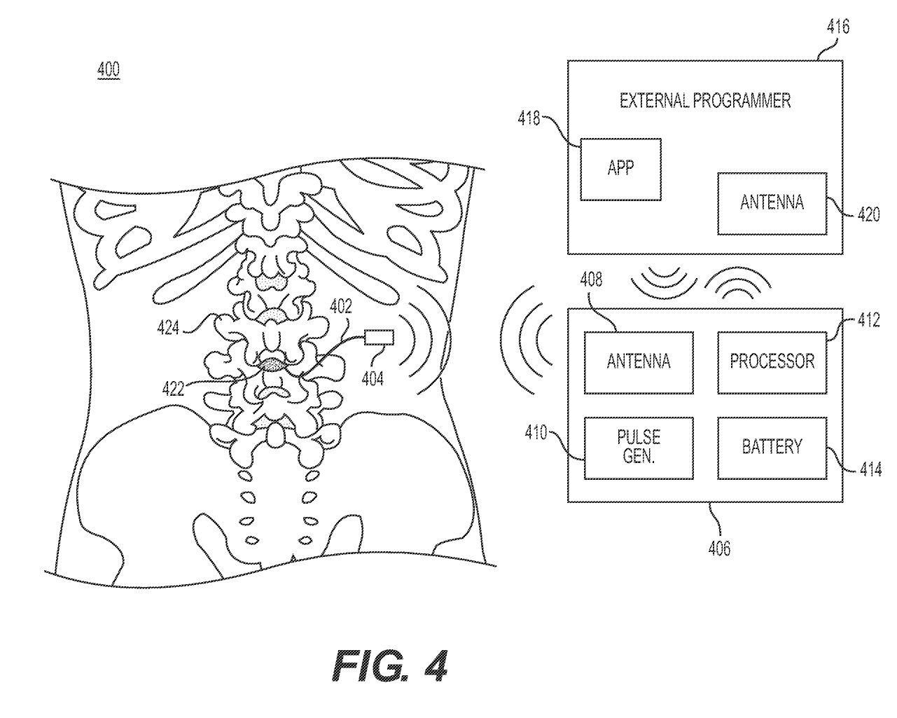

FIG. 4 illustrates an exemplary bone stimulator 400 consistent with the principles of this disclosure. Bone stimulator system 400 comprises an electrical implant 402 having a receiver 404. The electrical implant may be any kind of electrode. System 400 may also comprise an external pulse generator system 406 having an antenna 408, a pulse generator 410, and processor 412, and a battery 414. System 400 may also comprise an external programmer 416 having an embedded application 418 and an antenna 420.

In the exemplary embodiment depicted in FIG. 4, a patient may have undergone spinal fusion surgery on a fusion site 422 or spine 424. In addition to surgical procedures involved in fusing site 422, which may include but not limited to removing the vertebral disc and/or bone fragments and implanting a spacer and fixation devices, a surgeon may also implant electrical implant 402 at or near fusion site 422. Moreover, it is contemplated that electrical implant may alternatively be part of a spacer or other type of implant instead of being a separate component. In operation, electrical implant 402 may be an electrode that provides electromagnetic signals or pulses to affected area (fusion site 422) in order to promote the bone's healing process to promote fusion. Contrary to other types of stimulation systems as noted above, system 400 includes a relatively small electrical implant 402 with an incorporated receiver 404 that can be implanted by minimally invasive means such as a needle. The rest of the components (pulse generator 406 and external programmer 416) do not reside within the patient. This may eliminate more invasive types of procedures as described in other stimulation systems, and moreover, provide stimulation without any leads or components physically exiting the body in order to connect to the pulse generator. While FIG. 4 illustrates an example of during spinal fusion surgery, system 400 may be used in other applications involving mending fractured bones.

In operation, a user may interact with external programmer 416 to select certain therapy program(s). Different types of therapies may be available such that pulse generator 406 may provide a high frequency pulsed waveform, a low frequency pulsed waveform, a bursted pulse waveform, and/or a tonic stimulation. External programmer 416 may be a stand-alone device specifically configured to communicate with pulse generator system 406. External programmer 416 may be incorporated as part of a smartphone system with embedded application 418 and antenna 420 forming the basis for selecting therapy program(s) and communicating with pulse generator 406 through its antenna 408. Pulse generator 406, after receiving therapy instructions from external programmer 416, may wirelessly communicate with receiver 404 (via antenna 408) to deliver the electromagnetic signals or pulses for electrical implant 402. Electrical implant 402 may have been previously implanted at the time of surgery or afterwards through the use of a relatively small surgical needle. Pulse generator 410 may also contain a processor 412 that executes instructions received from the external programmer 416 and program pulse generator 410. Battery 414 may be a power source for pulse generator system 406 and may be re-chargeable using means known in the art.

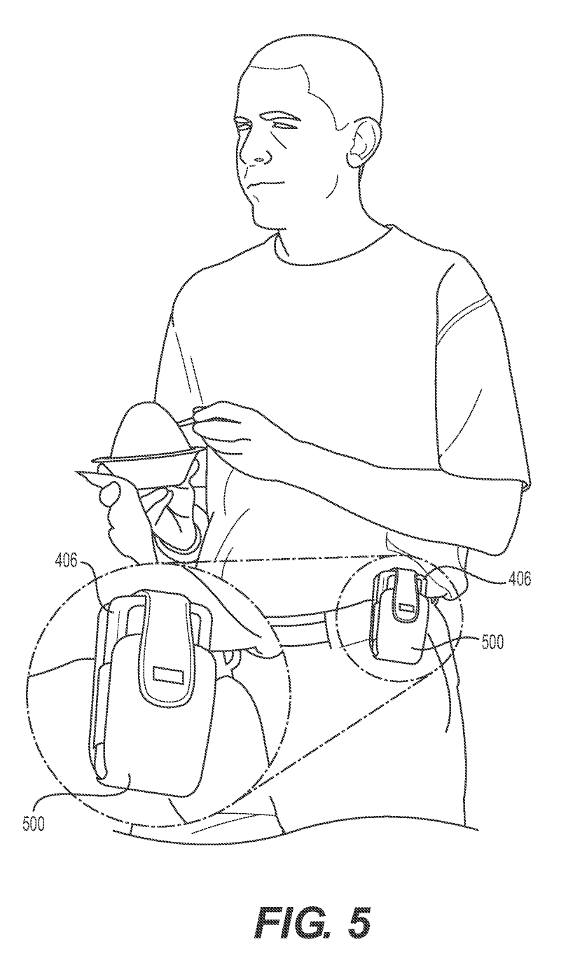

In order for pulse generator system 406 to communicate with electrical implant 402 via receiver 404, a certain distance should be maintained in order for proper wireless communication to occur. In order to facilitate such a connection, pulse generator system 406 may externally reside on a location on the patient near or adjacent to the internal location of electrical implant 402. For example, as shown in FIG. 5, pulse generator system 406 may be housed in a case 500, or any other position securing device or system, that may be worn by the user. In this example, a user may program pulse generator system 406 via a smartphone app and pulse generator system 406 may wirelessly deliver electromagnetic signals and/or pulses to electrical implant 402 inside the user.

Although the bone stimulator system has been described relative to an application involving spinal fusion, the principles apply to any type of procedure involving bone healing and/or fusion, such as a broken bone in another area of the body. The invention being thus described; it will be obvious that the same may be varied in many ways. Such variations are not to be regarded as a departure from the spirit and scope of the invention, and all such modifications as would be obvious to one skilled in the art are intended to be included within the scope of the following claims. Although individual embodiments are discussed herein, the invention covers all combinations of all those embodiments.

What is claimed is:

1. A bone stimulation system comprising:
an electrical implant comprising a receiver;
a pulse generator system comprising a pulse generator and a pulse generator antenna, wherein the pulse generator is in wireless communication with the receiver via the pulse generator antenna; and
a programmer comprising a programmer antenna, wherein the programmer is in wireless communication with the pulse generator system via the programmer antenna and the pulse generator antenna,
wherein the pulse generator system provides electromagnetic signals to the electrical implant via communication between the pulse generator antenna and the receiver.

2. The bone stimulator system of claim 1, wherein the programmer comprises one or more selectable therapy programs.

3. The bone stimulator system of claim 2, wherein the pulse generator system further comprises a processor configured to execute instructions consistent with a selected therapy program to generate electromagnetic signals.

4. The bone stimulator system of claim 3, wherein the pulse generator delivers electromagnetic signals to the electrical implant consistent with the selected therapy program.

5. The bone stimulator system of claim 4, wherein the electrical implant is an electrode implanted at near a target bone.

6. The bone stimulator system of claim 5, wherein the pulse generator system resides outside of the patient's body.

7. The bone stimulator system of claim 6, wherein the programmer is external to the patient's body.

8. The bone stimulator system of claim 7, wherein the programmer is a smartphone comprising an embedded application configured to the select one or more therapy programs.

9. The bone stimulator system of claim 8, wherein the pulse generator system is housed in a case external to the patient at a location near the electrical implant.

10. The bone stimulator system of claim 9, wherein the pulse generator further comprises a battery configured to be a power source for the pulse generator system.

11. A bone stimulation system comprising;
an implantable electrode having a receiver;
an external pulse generator system comprising a pulse generator, a processor, a pulse generator antenna, and a battery, wherein the pulse generator is in wireless communication with the receiver via the pulse generator antenna; and
an external programmer comprising a programmer antenna, wherein the programmer is in wireless communication with the pulse generator system via the programmer antenna and the pulse generator antenna,
wherein the programmer comprises one or more selectable therapy programs and wherein the pulse generator system provides electromagnetic signals representative of a selected therapy program to the implantable electrode via communication between the pulse generator antenna and the receiver.

12. The bone stimulator system of claim 11, wherein the external programmer comprises one or more selectable therapy programs.

13. The bone stimulator system of claim 12, wherein the processor is configured to execute instructions consistent with a selected therapy program to generate electromagnetic signals.

14. The bone stimulator system of claim 13, wherein the pulse generator delivers electromagnetic signals to the electrical implant consistent with the selected therapy program.

15. The bone stimulator system of claim 14, wherein the electrode is configured to be implanted at near a target bone.

16. The bone stimulator system of claim 15, wherein the battery is configured to be a power source for the pulse generator system.

17. The bone stimulator system of claim 16, wherein the battery is rechargeable.

18. The bone stimulator system of claim 17, wherein the external programmer is a smartphone comprising an embedded application configured to select one or more of the therapy programs.

19. The bone stimulator system of claim 18, wherein the external pulse generator system is housed in a case external to the patient at a location near the electrode.

20. The bone stimulator system of claim 19, the electromagnetic signals comprising electromagnetic pulses.
